(19) United States
(12) Patent Application Publication
(10) Pub. No.: US 2002/0052029 A1
(43) Pub. Date: May 2, 2002

(54) Method for preparing water-insoluble alpha-1, 4-glucans

(76) Inventors: Martin Quanz, Berlin (DE); Nicholas Provart, San Diego, CA (US); Ronald Banasiak, Potsdam (DE)

Correspondence Address: Gilberto M. Villacorta, Ph.D., PEPPER HAMILTON LLP, Hamilton Square, 600 14th Street, N.W., Suite 500, Washington, DC, 20005, US

(21) Appl. No.: 09/740,824

(22) Filed: Dec. 21, 2000

Related U.S. Application Data
(63) Continuation of application No. PCT/EP99/04199, filed on Jun. 17, 1999.

(30) Foreign Application Priority Data
Jun. 24, 1998 (DE) 198 27 978.1

(51) Int. Cl. C12P 19/22

(52) U.S. Cl. 435/95

(57) ABSTRACT

An in-vitro method for producing water-insoluble α-1,4-glucan is described, wherein saccharose is reacted in a buffer-free system using an amylosaccharase.

### DESCRIPTION

The present invention relates to an in-vitro method for preparing water-insoluble α-1,4-glucans in a buffer-free system.

There is great industrial interest in biotechnological methods for preparing polysaccharides, in particular water-insoluble α-1,4-glucans which are not accessible, or are only accessible with great difficulty, to pathways of classical organic synthesis pathways. However, for cost reasons only a few of these methods have been brought to commercial utilization to date. Biotechnological methods have advantages over the classical route of organic chemical synthesis. Thus enzyme-catalyzed reactions generally proceed with much higher specificities (regiospecificity, stereospecificity,) at higher reaction rates, under milder reaction conditions and lead to higher yields These factors are of outstanding importance in the preparation of novel polysaccharides.

Biotransformations, that is to say the in-vitro conversion of substances by purified or partially purified enzymes offer further advantages in comparison with biotechnological in-vivo methods Compared with the in-vivo methods they are distinguished by improved controllability and a greater reproducibility, since the reaction conditions in vitro can be set in a defined manner, in contrast to the conditions in a living organism This makes it possible to prepare constant products of great uniformity and purity and thus of high quality, which is of great importance for further industrial use. The workup of products of constant quality leads to reductions in costs, because the process parameters which are required for the workup do not need to be optimized again for each workup batch. A further advantage of in-vitro methods is that the products, in contrast to in-vivo methods, are free per se from the organisms. This is absolutely necessary for certain applications in the food industry and in the pharmaceutical industry. In order to be able to utilize the advantageous properties of water-insoluble α-1,4-glucans on an industrial scale, there is an urgent requirement for them to be provided inexpensively. On an industrial scale, to date, only water-soluble α-1,4-glucans, for example in the form of amylose, have been accessible. To prepare water-insoluble α-1,4-glucans, to date in the patent application WO 95/31553 and in Remaud-Simon et al. (Remaud-Simon, in Petersen, Svenson and Pedersen (Eds.) Carbohydrate bioengineering: Elsevier Science B. V., Amsterdam, The Netherlands (1995), pp. 313-320) a method using an amylosucrase from Neisseria polysaccharea has been described. This in-vitro method is based on the conversion of sucrose to α-1,4-glucans and fructose using a partially purified amylosucrase and is carried out in a sodium citrate buffer (pH 6.5) or a sodium maleate buffer (pH 6.4). The following reaction mechanism was postulated in WO 95/31553:

sucrose+(α-1,4-glucan)ₙ→fructose+(α-1,4-glucan)ₙ₊₁

On the basis of this reaction scheme, linear oligomeric or polymeric α-1,4-glucans serve as acceptors for a chain-extending reaction which leads to water-insoluble α-1,4-glucan polymers. In contrast to WO 95/31553, Remaud-Simon et al. (supra) additionally used 0.1 g/l of glycogen as an exogenous polysaccharide acceptor. This branched polysaccharide acceptor led to an increase in the reaction rate compared with the biotransformation in the absence of an exogenous polysaccharide acceptor.

The systems described to date for preparing polyglucans using amylosucrases proceed in buffered aqueous solutions. Not all of these methods yield water-insoluble α-1,4-glucans, The use of buffer chemicals and the working time required to establish the required buffer conditions lead to considerable process costs and thus make the commercial use of these systems more difficult. Further costs are produced by purification steps which are required in order to remove residues of the buffer salts from the biotransformation products (α-1,4-glucans and fructose). This is of great importance especially when these products are used in the food and pharmaceutical industries. There is therefore a need for methods for the efficient preparation of water-insoluble α-1,4-glucans which is commercially utilizable and leads to high-purity products.

The object thus underlying the present invention is to provide a method which is suitable for the industrial preparation of water-insoluble α-1,4-glucans which also leads to high-purity products.

This object is achieved by the provision of the embodiments featured in the patent claims.

The present invention thus relates to a method for preparing water-insoluble α-1,4-glucans in which sucrose is converted to water-insoluble α-1,4-glucans and fructose by an enzyme having the enzymatic activity of an amylosucrase, which comprises carrying out the conversion in an aqueous, buffer-free system.

It has surprisingly been found that, for the in-vitro preparation of water-insoluble α-1,4-glucans by an amylosucrase from Neisseria polysaccharea, an aqueous buffer-free system can be used. The efficiency of this method which can be determined on the basis of fructose release or sucrose consumption, corresponds to that of the buffered system. This is surprising, because the functionality of enzymes used could only previously be detected in buffered solutions (MacKenzie et al., Can. J. Microbiol. 23 (1977), 1303-1307; Okada and Hehre, J. Biol. Chem. 249 (1974), 126-135; Tao et al., Carbohydrate Res. 182 (1988), 163-174; Butcher et al., J. Bacteriol. 179 (1997), 3324-3330; WO 95/31553).

The inventive method now makes possible a great reduction in costs of the in-vitro preparation of insoluble α-1,4-glucans. In particular the following are avoided: working steps and apparatuses connected with the preparation of buffer solutions and also with the setting and if appropriate maintenance of the pH. A further decisive advantage of the inventive method is also the increased degree of purity of the products, which is of great importance especially for applications in the food sector and in the food, cosmetics and pharmaceutical industries. The buffer-free system also offers the advantage that the products contain no residues of buffer salts. Complex purification steps for removing these salts which would interfere in certain applications in the food and pharmaceutical industries are therefore not required. This leads to a further great reduction in costs. In addition to the water-insoluble α-1,4-glucans, in the inventive method fructose is formed. This can be used for the inexpensive production of “high fructose syrups” (HFS). The inventive method, owing to the buffer-free reaction conditions, leads to products of high purity. Complex purification of the fructose is therefore not necessary, in contrast to conventional methods for HFS preparation from cornstarch which comprise costly process steps for removing the buffer salts by ion exchange (Crabb and Mitchinson, TIBTECH 15 (1997), 349-352).

An “in-vitro conversion” for the purposes of the present invention is a conversion, that is to say a reaction, which proceeds outside a living organism. “In vitro” means in particular that the inventive method takes place in a reaction vessel.

An enzyme having the enzymatic activity of an amylosucrase (EC. 2.4.1.4.) is taken to mean an enzyme which catalyzes the following reaction:

sucrose+(α-1,4-glucan)ₙ→fructose+(α-1,4-glucan)ₙ₊₁

The enzymatic activity of an amylosucrase can be detected, for example, as described in the examples of the present application.

In the context of the present invention, an amylosucrase is also taken to mean an enzyme which, starting from sucrose and branched polysaccharide acceptors, for example glycogen, amylopectin or dextrin, catalyzes the synthesis of sucrose and linear α-1,4-glucan chains on these polysaccharide acceptors. That is to say the amylosucrase catalyzes an α-1,4-glucan chain extension on these branched acceptors also. The resultant products, in comparison with the branched starting materials used, have a lower degree of branching. These products also are termed water-insoluble α-1,4-glucans in the context of the present invention.

In principle, in the inventive method, any amylosucrase can be used. Preferably, an amylosucrase of prokaryotic origin is used. Enzymes of this type are, for example, known from Neisseria perflava (Okada and Hehre, J. Biol Chem. 249 (1974), 126-135; Mackenzie et al, Can. J. Microbiol. 23 (1977), 1303-1307) or Neisseria canis, Neisseria cinerea, Neisseria denitrificans, Neisseria sicca and Neisseria subflava (MacKenzie et al, Can. J. Microbiol. 24 (1972, 357-362). In addition, WO 95/31553 describes an amylosucrase from Neisseria polysaccharea. Particularly preferably, an amylosucrase naturally secreted by a prokaryote is used.

In a preferred embodiment of the inventive method, an amylosucrase from a bacterium of the genus Neisseria is used, particularly preferably an amylosucrase from the species Neisseria polysaccharea.

For the purposes of the invention, water-insoluble α-1,4-glucans are the polysaccharides prepared by the above-described conversion of sucrose using an amylosucrase. The term “water-insoluble glucans” is taken to mean in particular the polysaccharides prepared by the above-described conversion of sucrose using an amylosucrase which, according to the definition of the German Pharmacopeia (DAB=Deutsches Arzneimittelbuch, Wissenschaftliche Verlagsgesellschaft mbH, Stuttgart, Govi-Verlag GmbH, Frankfurt, 9th edition, 1987), come under the category of “sparingly soluble” compounds, “very sparingly soluble” or “virtually insoluble” compounds.

For the purposes of the present invention the term “buffer-free system” is an aqueous system which contains essentially no buffer salts. The term “buffer salts” is taken to mean in this context inorganic and organic salts, in particular salts of weak acids and bases. The term “essentially no” is taken to mean in this context buffer salt concentrations of a maximum of 25 mm, in a preferred embodiment a maximum of 10 mm, in a further preferred embodiment a maximum of 5 mm and in a very particularly preferred embodiment a maximum of 1 mm.

In a further particularly preferred embodiment of the inventive method, an aqueous system can be used which contains inorganic and organic salts only in trace amounts (<1 mm) as impurity. Very particularly preferably the aqueous buffer-free system is pure water.

In a particularly preferred embodiment of the inventive method, a purified amylosucrase is used. A purified amylosucrase here is taken to mean an enzyme which is substantially free from cell constituents of the cells in which the protein is synthesized. Preferably, the term “purified amylosucrase” means an amylosucrase which has a purity of at least 80%, preferably at least 90%, and particularly preferably at least 95%.

The use of a purified protein for preparing α-1,4-glucans offers various advantages. In comparison with methods which operate using partially purified protein extracts, the reaction medium of the inventive method contains no residues of the production strain (microorganism) which is used for purification or biotechnological production of the protein.

Furthermore, by using the purified protein, advantages can be seen for application in the food and pharmaceutical industries. Owing to the defined reaction medium composition, which is free from all unnecessary constituents, the product's constituents are also defined more precisely. This leads to a considerably less extensive approval procedure for these products produced by biotechnology in the food and pharmaceutical industries, in particular because these products should have no traces of a transgenic microorganism.

In a particularly preferred embodiment of the inventive method, the amylosucrase is a protein produced as a recombinant. In the context of the present invention this is taken to mean a protein which was produced by introducing a DNA sequence coding for the protein into a host cell and expressing it there. The protein can then be isolated from the host cell and/or from the culture medium. The host cell in this case is preferably a bacterium or a protist (for example fungi, in particular yeast, algae) as defined, for example in Schlegel “Allgemeine Mikrobiologie” [General Microbiology] (Georg Thieme Verlag, 1985, 1-2). Particularly preferably, the amylosucrase is secreted by the host cell. Host cells of this type for the production of a recombinant arylosucrase can be produced by methods known to those skilled in the art.

A review of various expression systems may be found, for example, in Methods in Enzymology 153 (1987), 385-516 and in Bitter et al. (Methods in Enzymology 153 (1987), 516-544). Expression vectors are described to a great extent in the literature. In addition to a selection marker gene and a replication origin ensuring replication in the selected host, they generally contain a bacterial or viral promoter, and usually a termination signal for the transcription. Between the promoter and the termination signal are situated at least one restriction site or a polylinker which enables the insertion of a coding DNA sequence. The promoter sequence used can, if it is active in the selected host organism, be the DNA sequence naturally controlling the transcription of the corresponding gene. However, this sequence can also be replaced by other promoter sequences. Either promoters can be used which cause constitutive expression of the gene, or inducible promoters can be used which permit specific regulation of the expression of the following gene. Bacterial and viral promoter sequences having these properties are extensively described in the literature. Regulatory sequences for expression in microorganisms (for example E. coli, S. cerevisiae) are adequately described in the literature. Promoters which permit particularly high expression of the following gene are, for example, the T7 promoter (Studier et al., Methods in Enzymology 185 (1990), 60-89), lacuv5, trp, trp-lacUV5 (DeBoer et al., in Rodriguez and Chamberlin (Eds), Promoters, Structure and Function; Praeger, N.Y., (1982), 462-481; DeBoer et al., Proc. Natl. Acad. Sci. USA (1983), 21-25), 1p1, rac (Boros et al., Gene 42 (1986), 97-100). Generally, the amounts of protein reach their maximum from the middle to toward the end of the logarithmic phase of the growth cycle of the microorganisms. For the synthesis of proteins, therefore, preferably inducible promoters are used. These frequently lead to higher yields of protein than constitutive promoters. The use of strong constitutive promoters frequently leads, via the constant transcription and translation of a cloned gene, to energy for other essential cell functions being lost and thus cell growth being retarded (Bernard R Glick/Jack J. Pasternak, Molekulare Biotechnologie (1995), Spektrum Akademischer verlag GmbH, Heidelberg Berlin Oxford, p. 342). To achieve an optimum amount of protein, therefore, frequently a two-step method is employed. Firstly, the host cells are cultured to a relatively high cell density under optimal conditions. In the second step the transcription is then induced depending on the type of promoter used. A particularly suitable promoter in this context is a tac promoter (DeBoer et al., Proc. Natl. Acad. Sci. USA 80 (1983), 21-25) which can be induced by lactose or TPTG (=isopropyl-β-D-thiogalactopyranoside). Termination signals for the transcription are also described in the literature.

The host cell can generally be transformed using the amylosucrase-coding DNA by standard methods, as described, for example, in Sambrook et al. (Molecular Cloning: A Laboratory Course Manual, 2nd edition (1989), Cold Spring Harbor Press, New York). The host cell is cultured in nutrient media which meet the requirements of the respective host cell used, in particular taking into account the pH, temperature, salt concentration, aeration, antibiotics, vitamins, trace elements etc.

The enzyme produced by the host cells can be purified by conventional purification methods, such as precipitation, ion-exchange chromatography, affinity chromatography, gel filtration, reversed-phase HPLC etc.

By modification of the DNA which is expressed in the host cells and codes for an amylosucrase, a polypeptide may be produced in the host cell which, owing to certain properties, can be isolated more readily from the culture medium. Thus, there is the possibility of expressing the protein to be expressed as a fusion protein with a further polypeptide sequence whose specific binding properties enable the fusion protein to be isolated via affinity chromatography (e.g. Hopp et al., Bio/Technology 6 (1988), 1204-1210; Sassenfeld, Trends Biotechnol. 8 (1990), 88-93).

In a preferred embodiment of the inventive method, an amylosucrase is used which is produced as a recombinant and was secreted by the host cell into the nutrient medium, so that cell digestion and further purification of the protein is not necessary, because the secreted protein can be isolated from the supernatant. To remove residual constituents of the culture medium, methods customary in process engineering, for example dialysis, reverse osmosis, chromatographic methods etc., can be used. The same also applies to concentrating the protein secreted into the culture medium. The secretion of proteins by microorganisms is usually mediated by N-terminal signal peptides (signal sequence, leader peptide). Proteins having this signal sequence can penetrate the cell membrane of the microorganism. Secretion of proteins can be achieved by the DNA sequence which codes for this signal peptide being joined to the corresponding amylosucrase-coding region. Preferably, the signal peptide is the natural signal peptide of the amylosucrase expressed, particularly preferably that of the amylosucrase from Neisseria polysaccharea.

Very particularly preferably, the signal peptide is that of the α-CGTase from Klebsiella oxytoca M5A1 (Fiedler et al., J. Mol. Biol. 256 (1996), 279-291) or a signal peptide as coded by nucleotides 11529-11618 of the sequence accessible in GenBank under the access number X864014.

Alternatively, the asmylosucrace used in the inventive method can also have been produced using an in-vitro transcription and translation system which leads to the expression of the protein, without use of microorganisms.

In a preferred embodiment, in the inventive method, an external carbohydrate acceptor is added in the conversion of the sucrose by the amylosucrase. For the purposes of the present invention, an external carbohydrate acceptor is a molecule which is able to increase the initial rate of the conversion of sucrose by the amylosucrase. Preferably, the external carbohydrate acceptor is added to the reaction mixture at the beginning of the conversion. The use of external acceptors leads to a reduction of the process time and thus to a decrease in costs of the process. The carbohydrate acceptor is preferably an oligosaccharide or polysaccharide, preferably a linear polysaccharide, and particularly preferably a branched polysaccharide, for example dextrin, glycogen or amylopectin. If a α-1,4-glucan chain extension takes place on these acceptors, products are formed which, compared with the branched starting material, have a considerably lower degree of branching. The extent of the reduction of degree of branching depends in this case on the degree of polymerization n. If sucrose is used in a great molar excess compared with the acceptor, in the product α-1,6-branches can no longer be measured by methylation analysis (degree of branching <1%). These products are also termed water-insoluble α-1,4-glucans in the context of the present invention.

In a further preferred embodiment, the enzyme having the enzymatic activity of an amylosucrase is immobilized on a support material. Immobilization of the amylosucrase offers the advantage that the enzyme, as catalyst of the synthesis reaction, can be recovered from the reaction mixture in a simple manner and used repeatedly. Since the purification of enzymes is in general cost-intensive and time-consuming, immobilization and reuse of the enzyme makes considerable cost savings possible. A further advantage is the purity of the reaction products which do not contain protein residues.

A multiplicity of support materials are available for the immobilization of proteins, coupling to the support material being able to take place via covalent or noncovalent bonds (for a review see: Methods in Enzymology 135, 136, 137). Materials which are widely used as support materials are, for example, agarose, alginate, cellulose, polyacrylamide, silica or nylon.

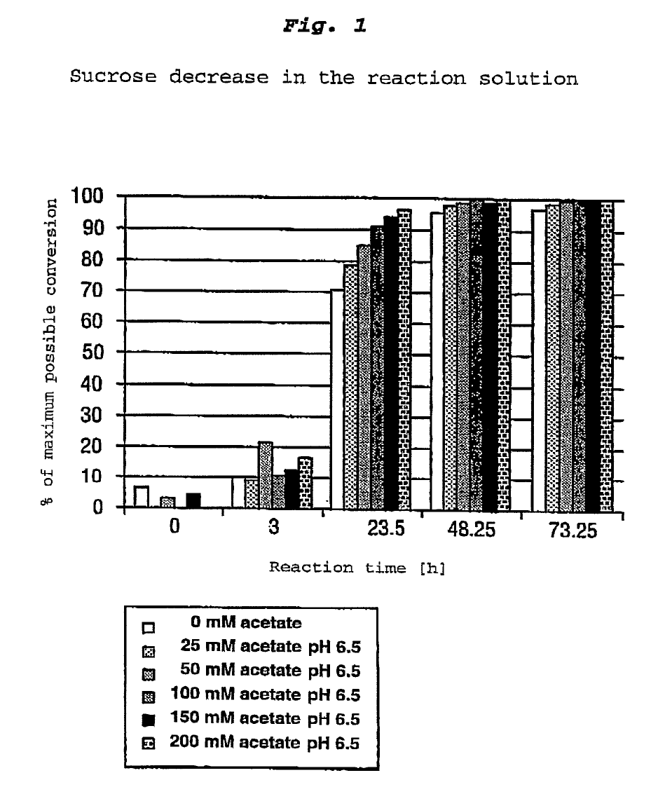

FIG. 1 shows a comparison of the efficiency of the in-vitro preparation of water-insoluble α-1,4-glucans by amylosucrase from Neisseria polysaccharea using different buffer salt concentrations. The efficiency of the method was determined on the basis of reduction in the amount of sucrose.

The examples below illustrate the invention.

### EXAMPLE 1

Purification of Amylosucrase

To produce an amylosucrase, E. coli cells were used which had been transformed using an amylosucrase from Neisseria polysaccharea (see WO 9531553). The DNA originated from an N. polysaccharea genome library.

An overnight culture of these E. coli cells which secrete the amylosucrase from Neisseria polysaccharea was centrifuged and resuspended in about 1/20 volume of 50 mM sodium citrate buffer (pH 6.5), 10 mM DTT (dithiothreitol), 1 mM PMSF (phenylmethylsulfonyl fluoride). The cells were then disintegrated twice using a French press at 16000 psi. Then 1 mM of MgCl₂ and Benzonase (from Merck; 100000 units; 250 units μl⁻¹) were added to the cell extract in a final concentration of 12.5 units ml⁻¹. The solution was then incubated for at least 30 min at 37° C. with gentle stirring. The extract was allowed to stand on ice for at least 1.5 hours. The extract was then centrifuged for 30 min at 4° C. at approximately 40000 g until the supernatant was relatively clear. Prefiltration of a PVDF membrane (millipore “Durapore”, or similar) which had a pore diameter of 0.45 μm was carried out. The extract was allowed to stand overnight at 4° C. Before carrying out the hydrophobic interaction (HI) chromatography, solid NaCl was added to the extract and a concentration of 2M NaCl was established. The extract was then again centrifuged for 30 min at 4° C. and approximately 40000 mg. The extract was then freed from the final residues of E. coli by filtering it with a PVDF membrane (millipore “Durapore”, or the like) which had a pore diameter of 0.22 μm. The filtered extract was separated on a butylsepharose-4B column (Pharmacia) (column volume: 93 ml, length: 17.5 cm). Approximately 50 ml of extract having amylosucrase activity of 1 to 5 units μl⁻¹ were applied to the column. Non-binding proteins were then washed from the column with 150 ml of buffer B (buffer B: 50 mM sodium citrate pH 6.5, 2 M NaCl). The amylosucrase was finally eluted using a falling linear NaCl gradient (from 2 M to 0 M NaCl in 50 mM sodium citrate in a volume of 433 ml at an inflow rate of 1.5 ml min⁻¹), which was generated using an automatic pump system (FPLC, Pharmacia). The amylosucrase is eluted between 0.7 M and 0.1 M NaCl. The fractions were collected, desalted via a PD10 Sephadex column (Pharmacia), stabilized with 8.7% of glycerol, tested for amylosucrase activity and finally frozen in storage buffer (8.7% glycerol, 50 mM citrate).

### EXAMPLE 2

Determination of Amylosucrase Activity

Purified protein or crude protein extract is incubated at 37° C. at various dilutions in 1 ml batches containing 5% sucrose, 0.1% glycogen and 100 mM citrate pH 6.5. After 5 min, 10 min, 15 min, 20 min, 25 min and 30 min, 10 μl were taken from each of these solutions and the enzymatic activity of amylosucrase was terminated by immediate heating to 95° C. In a coupled photometric test, the content of fructose released by the amylosucrase is determined. For this, 1 μl to 10 μl of the inactivated sample is added to 1 ml of 50 mM imidazole buffer pH 6.9, 2 mM MgCl₂, 1 mM ATP, 0.4 mM NAD and 0.5 U/ml of hexokinase. After sequential addition of glucose-6-phosphate dehydrogenase (from Leuconostoc mesenteroides) and phosphoglucose isomerase, the change in absorption at 340 nm is measured. Then, using the Lambert-Beer law, the amount of fructose released is calculated.

If the value obtained is related to the sampling time, the number of units (1 U=1 μmol fructose/min) (per μl of protein extract or μg of purified protein) may be determined.

### EXAMPLE 3

Reaction in the Buffer-free System Compared With the Buffered System

| | |
|---|---|
| Solution volumes: | 50 ml |
| Enzyme activity: | 5 units/ml |
| Buffer: | Na acetate pH 6.5, varied between |
| | 0 mM (= water) and 200 mM (Merck) |
| Substrate: | 10% sucrose (ICN) |
| Primer: | 0.1% dextrin, type IV potato |
| | (sigma) |

Procedure:

Solutions each of 50 ml reaction volume containing 10% sucrose, 0.1% dextrin, 250 units of amylosucrase and differing concentrations of a reaction buffer (25 mM, 50 mM, 100 mM or 200 mM Na acetate, pH 6.5) were incubated at 37° C. for 46 h and 73.25 h. in addition, a reaction mixture was made up without buffer, that is to say in demineralized water (pH 7.0). Except for the buffer substance, this reaction solution contained all of the abovementioned components.

To determine the conversion of sucrose to amylose and fructose, 1 ml aliquots were taken from each of the six reaction solutions at various points in time. The reaction was stopped in the samples taken by heating to 95° C. for 10 minutes. The conversion rates were determined by measuring the fructose formed or by determining the concentration of sucrose still present in the inactivated samples using a coupled enzymatic test in the photometer.

Enzyme assay:

| | |
|---|---|
| Assay volume: | 1 ml |
| Enzymes: | hexokinase from yeast, phospho- |
| | glucose isomerase, glucose- |
| | 6-phosphate dehydrogenase from |
| | Leuconostoc mesenteroides β-fructo- |
| | sidase from yeast (all enzymes: |
| | Boehringer Mannheim) |
| Assay buffer: | 1 mM ATP |
| | 0.4 mM NAD⁺ |
| | 50 mM imidazole pH 6.9 |

The test is based on the conversion of fructose to glucose-6-phosphate using hexakinase and phosphoglucose isomerase. The glucose-6-phosphate is then converted via glucose-6-phosphate dehydrogenase to 6-phosphogluconate. This reaction is linked to the conversion of NAD⁺ to NADH+H⁺, which can be measured photometrically at a wavelength of 340 nm. Using the Lambert-Beer law, the amount of fructose can be calculated from the resulting absorptions.

To determine the concentration of sucrose, β-fructosidase is added to the sample to be determined, in addition to the above-described reaction mixture.

This enzyme cleaves the sucrose into fructose and glucose. The concentration of the two monosaccharides resulting from this reaction are then determined as described above using the conversion of NAD⁺ to NADH+H⁺. The sucrose concentration can be calculated from the total of monosaccharides determined.

Result:

After approximately 73 h, under all reaction conditions the sucrose present in the reaction solution has been approximately 100% converted to axylose and fructose.

1. A method for preparing water-insoluble α-1,4-glucans in which sucrose is converted in vitro to water-insoluble α-1,4-glucans and fructose by an enzyme having the enzymatic activity of an arylosucrase, which comprises carrying out the reaction in an aqueous, buffer-free system.

2. The method as claimed in claim 1, wherein the amylosucrase is an enzyme from a prokaryotic organism.

3. The method as claimed in claim 2, wherein the prokaryotic organism belongs to the genus Neisseria.

4. The method as claimed in claim 3, wherein the prokaryotic organism is Neisseria polysaccharea.

5. The method as claimed in one of claims 1 to 4, wherein the amylosucrase is produced as a recombinant.

6. The method as claimed in one of claims 1 to 5, wherein a purified amylosucrase is used.

7. The method as claimed in one of claims 1 to 6, wherein the amylosucrase is bound to a support material.

8. The method as claimed in one of claims 1 to 7, wherein an external carbohydrate acceptor is added.
